(19) United States
(12) Patent Application Publication
(10) Pub. No.: US 2025/0020860 A1
(43) Pub. Date: Jan. 16, 2025

(54) LIGHT DIFFUSION DEVICE

(71) Applicant: FURUKAWA ELECTRIC CO., LTD., Tokyo (JP)

(72) Inventors: Hideaki HASEGAWA, Tokyo (JP); Keisuke TAKE, Tokyo (JP); Masaki IWAMA, Tokyo (JP); Shun-ichi MATSUSHITA, Tokyo (JP)

(21) Appl. No.: 18/713,707

(22) PCT Filed: Nov. 24, 2022
(86) PCT No.: PCT/JP2022/043335

§ 371 Date: May 28, 2024

(30) Foreign Application Priority Data
Dec. 2, 2021 (JP) 2021-196511

(51) Int. Cl. G02B 6/02; A61N 5/06; A61N 5/067; F21V 8/00

(52) U.S. Cl. CPC G02B 6/02342; G02B 6/001; G02B 6/02319; A61N 5/0601; A61N 2005/063; A61N 5/067

(57) ABSTRACT

Provided is a light diffusion device capable of uniformly emitting light from the outer circumferential surface of a light-emitting part of an optical fiber. This light diffusion device 1 comprises an optical fiber 20 composed of a core 21 positioned on the radial center side and a clad 22 positioned on the outer circumferential side of the core 21, and emits laser light, which is incident from a proximal end section of the optical fiber 20, from the distal end side of the optical fiber 20, wherein: the optical fiber 20 has a light transmission part 20a which transmits the laser light incident from the proximal end section toward the distal end section, and a light-emitting part 20b which emits, from the outer circumferential surface, the laser light transmitted from the light transmission part 20a by removing a portion positioned on the outer circumferential side of the clad 22 on the distal end side; and the maximum thickness of the clad 22 in the light-emitting part 20b is smaller than the thickness of the clad 22 in the light transmission part 20a.

### TECHNICAL FIELD

The present invention relates to a light diffusion device for use in medical equipment.

### BACKGROUND ART

A conventional light diffusion device is known to include an optical fiber including a core located at a center in a radial direction and a cladding adjacent to an outer periphery of the core and to emit, from a tip and an outer peripheral surface of a tip side of the optical fiber, laser light that is incident from a base end portion of the optical fiber (for example, see Patent Document 1). The optical fiber of the conventional light diffusion device includes a light transmitting part that transmits the laser light that is incident from the base end portion, and a light emitting part that emits, from the tip side, the laser light transmitted through the light transmitting part.

In photoimmunotherapy, which is one therapeutic method for cancer, a tip side of an optical fiber of a light diffusion device is inserted into a human body and used to irradiate laser light onto a drug that is administered to the human body and has reached cancer cells.
Patent Document 1: Japanese Patent No. 5766609

### DISCLOSURE OF THE INVENTION

### Problems to be Solved by the Invention

In the conventional light diffusion device, cladding on a tip side of an optical fiber is partially removed to expose a core, enabling light to be emitted from an outer peripheral surface of a light emitting part. In this case, in the conventional light diffusion device, there is a large difference between the refractive index of the core in the light emitting part and the refractive index of air present around the outer periphery of the core, resulting in a stronger effect of confining light. For this reason, laser light transmitted through a light transmitting part has an emission intensity that is different at a portion where the core is exposed and a portion that is covered with the cladding, and it is difficult to uniformly emit the laser light from the outer peripheral surface of the light emitting part. The emission intensity of the laser light emitted from the light emitting part is limited throughout the entire light emitting part. Cladding that is deeply etched or roughened until the core is exposed results in an increase in the area of an interface between the outer peripheral surface of the core and the air. A thermal resistance of the core is generally higher at the interface between the outer peripheral surface of the core and air than in the bulk of the core, and as the area increases, the thermal resistance increases, and the amount of heat generated when the laser light is emitted increases, which is undesirable.

An object of the present invention is to provide a light diffusion device capable of uniformly emitting light from an outer peripheral surface of a light emitting part of an optical fiber.

### Means for Solving the Problems

A light diffusion device including: an optical fiber including a core located at a center in a radial direction and a cladding adjacent to an outer periphery of the core, the light diffusion device emitting, from a tip side of the optical fiber, light that is incident from a base end portion of the optical fiber. The optical fiber includes: a light transmitting part that transmits, toward a tip, the light that is incident from the base end portion; and a light emitting part that, due to removal of an outer peripheral portion of the cladding from the cladding in the tip side, emits, from an outer peripheral surface of the light emitting part, the light transmitted through the light transmitting part. The cladding in the light emitting part has a maximum thickness smaller than a thickness of the cladding in the light transmitting part.

The maximum thickness of the cladding in the light emitting part of the light diffusion device according to the present invention is smaller than the thickness of the cladding in the light transmitting part by at least a wavelength of the light transmitted through the light transmitting part.

The light emitting part of the light diffusion device according to the present invention is formed in at least a part of the outer peripheral surface of the tip side of the optical fiber in a circumferential direction.

The light emitting part of the light diffusion device according to the present invention is formed on a portion of 30% or more of the outer peripheral surface of the tip side of the optical fiber in the circumferential direction.

The light diffusion device according to the present invention has an uneven surface formed on the light emitting part along a circumferential direction. The thickness of the cladding in the light emitting part is such that a height difference between a portion where the uneven surface protrudes most toward the outer periphery and a portion where the uneven surface protrudes least toward the outer periphery is at most a wavelength of the light transmitted through the light transmitting part.

The cladding of the light diffusion device according to the present invention has a thickness of 1 μm or greater and 50 μm or less.

The light emitting part of the light diffusion device according to the present invention has a diameter of a minimum circumscribed circle that is a circle passing through a protruding point of an uneven surface formed across the outer peripheral surface in a circumferential direction. The diameter of the minimum circumscribed circle is smaller than a diameter of the light transmitting part by at least a wavelength of the light transmitted through the light transmitting part.

The light emitting part of light diffusion device according to the present invention has a diameter of a maximum inscribed circle that is the circle passing through a recessing point of an uneven surface formed across the outer peripheral surface in a circumferential direction. The diameter of the maximum inscribed circle is smaller than a diameter of the light transmitting part by at least a wavelength of the light transmitted through the light transmitting part.

The core in the light transmitting part of the light diffusion device according to the present invention has an outer diameter of 100 μm or greater and 1000 μm or less.

The cladding in the light transmitting part of the light diffusion device according to the present invention has an outer diameter of 102 μm or greater and 1100 μm or less.

The difference in refractive index between the core and the cladding of the light diffusion device according to the present invention is 2% or greater and 11% or less.

The optical fiber of the light diffusion device according to the present invention is formed from a resin member.

### Effects of the Invention

According to the present invention, the light transmitted through the light transmitting part can be reliably emitted from the outer peripheral surface of the light emitting part, and thus the light can be uniformly emitted from the outer peripheral surface of the light emitting part, enabling the efficiency of treatment with photoimmunotherapy to be improved. Regarding light emitting uniformity with the present technology, variation in the light emitting intensity with respect to a fiber longitudinal direction is suppressed to 22% or less, which is a level at which there is no problem from a practical standpoint. In the conventional light diffusion device, the cladding is removed until the core is exposed, and thus the area of the interface between the outer peripheral surface of the core and air increases, and the thermal resistance at the interface increases. For this reason, in the conventional light diffusion device, the amount of heat generated during emission of light increases, and in a case in which a heated optical fiber or a catheter, into which an optical fiber is inserted, comes into contact with the skin or the like of a patient who is to undergo photoimmunotherapy, damage or the like may occur in healthy cells, and pain and burden of the patient may increase. On the other hand, in the light diffusion device of the present invention, the area of the interface between the outer peripheral surface of the optical fiber and air is reduced by making the unevenness of the cladding interface smaller, and thus, the heat generated during light emitting is suppressed, and the burden on the patient can be reduced. When the emission intensity is about 800 mW, the heat generated at the light emitting part is 70 degrees or greater in the conventional light diffusion device, whereas the heat generated in the light diffusion device of the present invention is 60 degrees or less. Further, in the light diffusion device of the present invention, heat generated by the light diffusion device is predominantly due to heat generated by the tip of the optical fiber, no heat generated by a light emitting region on the outer peripheral surface of the optical fiber can be observed, and thus a region where heat is generated is limited, and countermeasures can be easily applied, which is preferable from a practical standpoint.

### BRIEF DESCRIPTION OF THE DRAWINGS

FIG. 1 is a schematic diagram of a light diffusion device according to a first embodiment of the present invention;

FIG. 2 is a cross-sectional view of a light transmitting part of an optical fiber according to the first embodiment of the present invention;

FIG. 3 is a cross-sectional view of a light emitting part of the optical fiber according to the first embodiment of the present invention;

FIG. 4 is a cross-sectional view of a main part of the light emitting part of the optical fiber according to the first embodiment of the present invention;

FIG. 5 is a cross-sectional view of a light emitting part of an optical fiber according to a second embodiment of the present invention; and

FIG. 6 is a cross-sectional view showing another example of the light emitting part of the optical fiber of the present invention.

### PREFERRED MODE FOR CARRYING OUT THE INVENTION

### First Embodiment

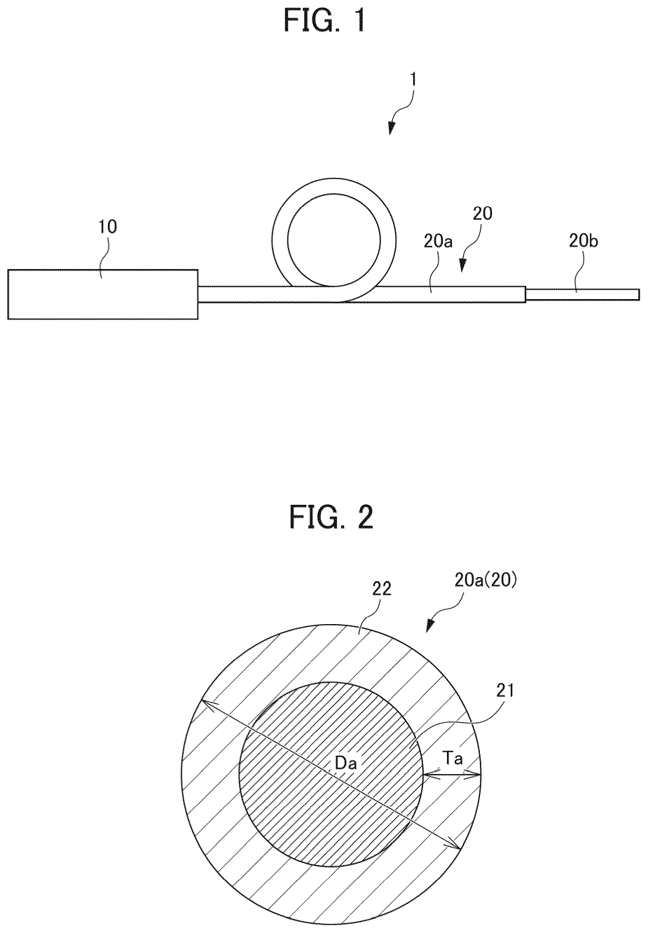

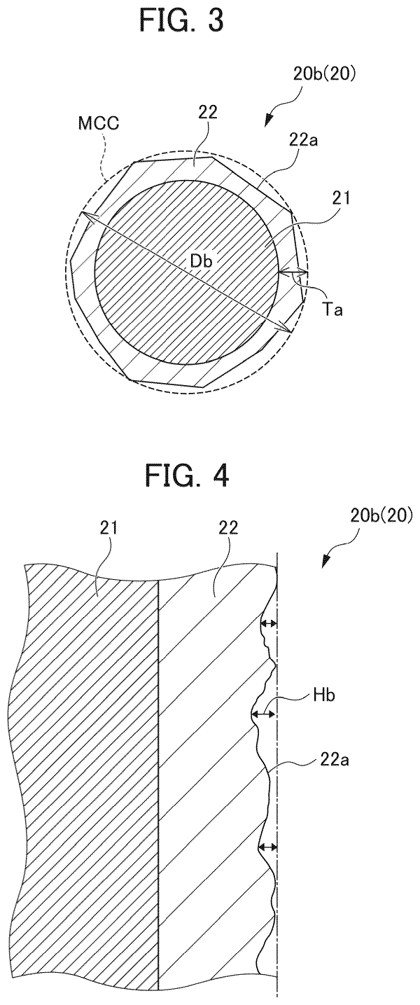

FIGS. 1 to 4 illustrate a first embodiment of the present invention. FIG. 1 is a schematic view of a light diffusion device, FIG. 2 is a cross-sectional view of a light transmitting part of an optical fiber, FIG. 3 is a cross-sectional view of a light emitting part of the optical fiber, and FIG. 4 is a cross-sectional view of a main part of the light emitting part of the optical fiber.

A light diffusion device 1 of the present embodiment is used in photoimmunotherapy, which is one method for treating cancer. In photoimmunotherapy, cancer is treated by administering, to a human body, a drug composed of antibodies that bind to cancer cells and a substance that reacts with light and irradiating the drug that has bonded to the cancer cells with laser light to destroy the cancer cells.

As illustrated in FIG. 1, the light diffusion device 1 includes a laser oscillator 10 as a light source for generating laser light, and an optical fiber 20 through which the laser light generated by the laser oscillator 10 is transmitted.

The laser oscillator 10 includes a semiconductor laser, generates laser oscillation by supplying electricity to the semiconductor laser, and generates laser light. The laser oscillator 10 generates red laser light having a wavelength of 670 nm or greater and 700 nm or less.

The optical fiber 20 is formed from a resin member. As illustrated in FIG. 2, the optical fiber 20 is a single-core optical fiber including a core 21 located at a center in a radial direction and a cladding 22 adjacent to an outer periphery of the core 21. The difference in refractive index between the core 21 and the cladding 22 of the optical fiber 20 is 2% or greater and 11% or less. For example, the optical fiber 20 has an outer diameter of 500 μm, the core 21 has an outer diameter of 480 μm, and the cladding 22 has a thickness of 10 μm. The cladding 22 of the optical fiber 20 preferably has an outer diameter of 102 μm or greater and 1100 μm or less. The core 21 of the optical fiber 20 preferably has an outer diameter of 100 μm or greater and 1000 μm or less. The cladding 22 preferably has a thickness of 1 μm or greater and 50 μm or less.

As illustrated in FIG. 1, the optical fiber 20 includes a light transmitting part 20a that transmits, toward a tip side, laser light that is incident from a base end portion, and a light emitting part 20b that, due to removal of an outer peripheral portion of the cladding 22 within a predetermined range in on the tip side, emits, from the outer peripheral surface, the laser light transmitted through the light transmitting part 20a.

The light emitting part 20b is formed in a range of, for example, 10 mm or more and 30 mm or less on the tip side of the optical fiber 20. The light emitting part 20b is formed by removing only the outer peripheral portion of the cladding 22 by, for example, etching while maintaining an inner peripheral portion of the cladding 22.

Due to the removal of the cladding 22, the size of the light emitting part 20b in the radial direction becomes smaller than a diameter Da of the light transmitting part 20a by at least the wavelength of the laser light, resulting in a change in the structure of the wavelength order in the longitudinal direction of the optical fiber 20 that causes a change in the light intensity distribution over a cross section of the optical fiber 20, which results in a light leak and the laser light being emitted from the outer peripheral surface. However, in the light emitting part 20b, it is difficult to uniformly remove the cladding 22 in a direction in which the outer peripheral surface extends and in the circumferential direction, resulting in partial variation. The above-described phenomenon in which light is emitted due to the change in the structure of the optical fiber 20 with respect to the longitudinal direction is caused by a mismatch between modes.

Therefore, as illustrated in FIG. 3, the outer diameter of the light emitting part 20b is defined as a diameter Db of a minimum circumscribed circle MCC that is a circle passing through a protruding point of an uneven surface 22a formed over the outer peripheral surface in a circumferential direction, and the light emitting part 20b is formed such that the diameter Db of the minimum circumscribed circle MCC is smaller than the diameter Da of the light transmitting part 20a by at least the wavelength of the laser light transmitted through the light transmitting part 20a.

Specifically, for example, in a case in which the diameter Da of the light transmitting part 20a is 500 μm and the wavelength of the laser light is 680 nm (0.68 μm), the light emitting part 20b is formed such that the diameter Db of the minimum circumscribed circle MCC of the light emitting part 20b is equal to or less than 499.32 μm.

The cladding 22 in the light emitting part 20b has a maximum thickness that is smaller than a thickness of the cladding 22 in the light transmitting part 20a. The maximum thickness of the cladding 22 in the light emitting part 20b is preferably smaller than the thickness of the cladding 22 in the light transmitting part 20a by at least the wavelength of the light transmitted through the light transmitting part 20a.

As illustrated in FIGS. 2 and 3, the average thickness Tb of the cladding 22 in the light emitting part 20b is preferably smaller than a thickness Ta of the cladding 22 in the light transmitting part 20a by at least the wavelength of the laser light transmitted through the light transmitting part 20a. Specifically, for example, in a case in which the thickness Ta of the cladding 22 in the light transmitting part 20a is 10 μm and the wavelength of the laser light is 680 nm (0.68 μm), the cladding 22 in the light emitting part 20b is formed such that the average thickness Tb thereof is equal to or less than 9.32 μm. With this configuration, a change in the structure of the wavelength order in the longitudinal direction of the optical fiber causes the light intensity distribution over a cross section of the optical fiber to change more significantly at the cladding portion, which results in more laser light being emitted from the outer peripheral surface of the light emitting part 20b.

Further, as illustrated in FIG. 4, for the uneven surface 22a that is formed along the circumferential direction of the cladding 22 in the light emitting part 20b, a height difference Hb between a portion where the uneven surface protrudes most toward the outer periphery and a portion where the uneven surface protrudes least toward the outer periphery is preferably at most the wavelength of the laser light transmitted through the light transmitting part 20a. By reducing local and fine structural changes in the cladding portion, more uniform emission characteristics can be realized. At the same time, the area of the interface between the outer peripheral surface of the light emitting part 20b and the air can be reduced, and thus the thermal resistance of the interface can be reduced, and heat generation can be reduced.

When used in photoimmunotherapy, the light diffusion device 1 configured as described above irradiates laser light onto a drug that has reached cancer cells while the tip side of the optical fiber 20 is inserted into the human body.

During irradiation, the laser light generated in the laser oscillator 10 propagates through the core 21 of the optical fiber 20 and is emitted from the light emitting part 20b located in the tip side of the optical fiber 20. The laser light emitted from the light emitting part 20b is uniformly emitted from the outer peripheral surface of the light emitting part 20b and is irradiated to a target site in the human body.

As described above, according to the light diffusion device 1 of the present embodiment, the light diffusion device 1 includes the optical fiber 20 including the core 21 located at the center in the radial direction and the cladding 22 adjacent to the outer periphery of the core 21, the light diffusion device 1 emits, from the tip side of the optical fiber 20, the laser light that is incident from the base end portion of the optical fiber 20. The optical fiber 20 includes: the light transmitting part 20a that transmits, toward the tip, the laser light that is incident from the base end portion; and the light emitting part 20b that, due to removal of an outer peripheral portion of the cladding from the cladding 22 in the tip side, emits, from the outer peripheral surface of the light emitting part 20b, the laser light transmitted through the light transmitting part 20a. The diameter Db of the light emitting part 20b, which is a minimum circumscribed circle MCC that is the circle passing through a protruding point of the uneven surface 22a formed over the outer peripheral surface in the circumferential direction, is smaller than the diameter Da of the light transmitting part 20a by a distance that is equal to or greater than a wavelength of the laser light transmitted through the light transmitting part 20a.

With this configuration, the laser light transmitted through the light transmitting part 20a can be reliably emitted from the outer peripheral surface of the light emitting part 20b, and thus the laser light can be uniformly emitted from the outer peripheral surface of the light emitting part 20b, enabling the efficiency of treatment with photoimmunotherapy improved.

The average thickness Tb of the cladding 22 in the light emitting part 20b is preferably smaller than a thickness Ta of the cladding 22 in the light transmitting part 20a by at least the wavelength of the laser light transmitted through the light transmitting part 20a.

With this configuration, the laser light transmitted through the light transmitting part 20a can be more reliably emitted from the outer peripheral surface of the light emitting part 20b, and the amount of the laser beam emitted from the outer peripheral surface of the light emitting part 20b can be increased.

The thickness of the cladding 22 in the light emitting part 20b is such that a height difference Hb between a portion where the uneven surface 22a protruding most toward the outer periphery and a portion where the uneven surface 22a protruding least toward the outer periphery is preferably at most the wavelength of the laser light transmitted through the light transmitting part 20a.

With this configuration, the laser light that is transmitted through the light transmitting part 20a can be uniformly emitted from the entire outer peripheral surface of the light emitting part 20b, and an emission amount of the laser light from the outer peripheral surface of the light emitting part 20b can be increased.

### Second Embodiment

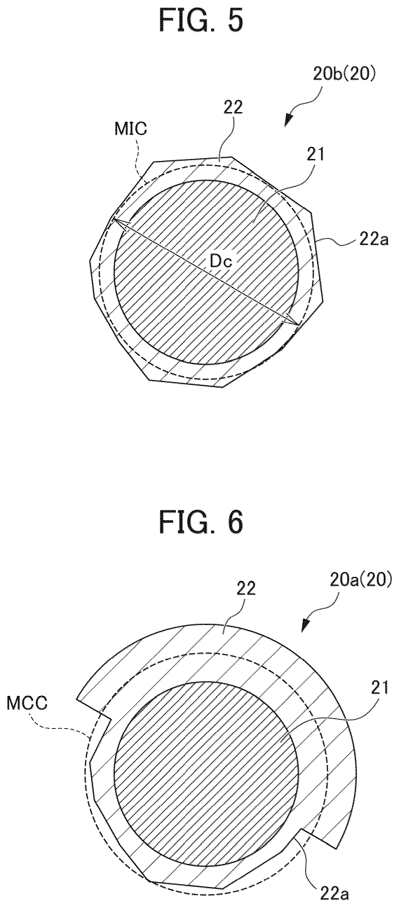

FIG. 5 is a cross-sectional view of a light emitting part of an optical fiber according to a second embodiment of the present invention.

The outer diameter of the light emitting part 20b of the light diffusion device 1 of the present embodiment is defined as the diameter Dc of the maximum inscribed circle MIC that is a circle passing through a recessing point of the uneven surface 22a formed over the outer peripheral surface in the circumferential direction, and the light emitting part 20b is formed such that the diameter Dc of the maximum inscribed circle MIC is smaller than the diameter Da of the light transmitting part 20a by at least the wavelength of the laser light transmitted through the light transmitting part 20a.

As described above, according to the light diffusion device 1 of the present embodiment, the light diffusion device 1 includes the optical fiber 20 including the core 21 located at the center in the radial direction and the cladding 22 adjacent to the outer periphery of the core 21, the light diffusion device 1 emits, from the tip side of the optical fiber 20, the laser light incident from the base end portion of the optical fiber 20. The optical fiber 20 includes: the light transmitting part 20a that transmits, toward the tip, the laser light that is incident from the base end portion and the light emitting part 20b that, due to removal of an outer peripheral portion of the cladding from the cladding 22 in the tip side, emits, from the outer peripheral surface of the light emitting part 20b, the laser light transmitted through the light transmitting part 20a. The light emitting part 20b has a diameter Dc of a maximum inscribed circle MIC that is the circle passing through a recessing point of the uneven surface 22a formed over the outer peripheral surface in the circumferential direction. The diameter Dc is smaller than a diameter Da of the light transmitting part 20a by a distance that is equal to or greater than a wavelength of the laser light transmitted through the light transmitting part 20a.

With this configuration, the laser light transmitted through the light transmitting part 20a can be reliably emitted from the outer peripheral surface of the light emitting part 20b, and thus the laser light can be uniformly emitted from the outer peripheral surface of the light emitting part 20b, enabling the efficiency of treatment with photoimmunotherapy to be improved.

It should be noted that although embodiments have been described in which the outer peripheral portion of the cladding 22 in the light emitting part 20b is removed over the outer periphery, the present invention is not limited thereto. As long as the optical fiber emits the laser light from the outer peripheral surface of the light emitting part 20b, as illustrated in FIG. 6, only a portion of the cladding 22 may be removed, in the circumferential direction, from a part of the outer periphery of the cladding 22 of the light emitting part 20b so that the laser light is emitted from only the part in the circumferential direction. In other words, the light emitting part may be formed on at least a part of the outer peripheral surface of the tip side of the optical fiber in a circumferential direction. The light emitting part may be formed in a portion of 30% or more of the outer peripheral surface of the tip side of the optical fiber in the circumferential direction, and may be formed, for example, in a range of 120 degrees to 180 degrees in the circumferential direction on the tip side of the optical fiber. The light emitting part may be formed discretely in the circumferential direction on the tip side of the optical fiber, and the total area of the light emitting part may be 30% or more of the area of the outer peripheral surface of the tip side of the optical fiber.

In the embodiments described above, a single-core optical fiber including one core 21 and the cladding 22 adjacent to the outer periphery of the one core 21 is illustrated, but the present invention is not limited thereto. In the case of a multicore optical fiber in which multiple cores are provided within one cladding, an outer peripheral portion of the one cladding may be removed so that laser light is emitted.

### EXPLANATION OF REFERENCE NUMERALS

1 light dispersion device
10 laser oscillator
20 optical fiber
20a light transmitting part
20b light emitting part
21 core
22 cladding
22a uneven surface
CMM minimum circumscribed circle
CIM maximum inscribed circle

1. A light diffusion device comprising:
an optical fiber including a core located at a center in a radial direction and a cladding adjacent to an outer periphery of the core, the light diffusion device emitting, from a tip side of the optical fiber, light that is incident from a base end portion of the optical fiber,
wherein the optical fiber includes: a light transmitting part that transmits, toward a tip, the light that is incident from the base end portion; and a light emitting part that, due to removal of an outer peripheral portion of the cladding from the cladding in the tip side, emits, from an outer peripheral surface of the light emitting part, the light transmitted through the light transmitting part, and
the cladding in the light emitting part has a maximum thickness smaller than a thickness of the cladding in the light transmitting part.

2. The light diffusion device according to claim 1,
wherein the maximum thickness of the cladding in the light emitting part is smaller than the thickness of the cladding in the light transmitting part by at least a wavelength of the light transmitted through the light transmitting part.

3. The light diffusion device according to claim 1,
wherein the light emitting part is formed in at least a part of the outer peripheral surface of the tip side of the optical fiber in a circumferential direction.

4. The light diffusion device according to claim 3,
wherein the light emitting part is formed on a portion of 30% or more of the outer peripheral surface of the tip side of the optical fiber in the circumferential direction.

5. The light diffusion device according to claim 1,
wherein the light emitting part has an uneven surface formed along a circumferential direction, and
the thickness of the cladding in the light emitting part is such that a height difference between a portion where the uneven surface protrudes most toward the outer periphery and a portion where the uneven surface protrudes least toward the outer periphery is at most a wavelength of the light transmitted through the light transmitting part.

6. The light diffusion device according to claim 1,
wherein the cladding has a thickness of 1 μm or greater and 50 μm or less.

7. The light diffusion device according to claim 1,
wherein the light emitting part has an uneven surface formed over the outer peripheral surface in a circumferential direction, and a diameter of a minimum circumscribed circle that is a circle passing through a protruding point of the uneven surface is smaller than a diameter of the light transmitting part by at least a wavelength of the light transmitted through the light transmitting part.

8. The light diffusion device according to claim 1,
wherein the light emitting part has an uneven surface formed over the outer peripheral surface in a circumferential direction, and a diameter of a maximum inscribed circle that is a circle passing through a recessing point of the uneven surface is smaller than a diameter of the light transmitting part by at least a wavelength of the light transmitted through the light transmitting part.

9. The light diffusion device according to claim 1,
wherein the core in the light transmitting part has an outer diameter of 100 μm or greater and 1000 μm or less.

10. The light diffusion device according to claim 1,
wherein the cladding in the light transmitting part has an outer diameter of 102 μm or greater and 1100 μm or less.

11. The light diffusion device according to claim 1,
wherein the core and the cladding have a difference in refractive index that is 2% or greater and 11% or less.

12. The light diffusion device according to claim 1,
wherein the optical fiber is formed from a resin member.
